(19) United States
(12) Patent Application Publication
(10) Pub. No.: US 2024/0374087 A1
(43) Pub. Date: Nov. 14, 2024

(54) ANTIBACTERIAL FLUID DISPENSING AND TEMPERATURE SCANNING DEVICE

(71) Applicants: Ray Peermal, Coral Springs, FL (US); Ryan V. Mahabir, South Ozone Park, NY (US); Shivendra Jairam, Toronto (CA); Prakash Mahabir, Coral Springs, FL (US)

(72) Inventors: Ray Peermal, Coral Springs, FL (US); Ryan V. Mahabir, South Ozone Park, NY (US); Shivendra Jairam, Toronto (CA); Prakash Mahabir, Coral Springs, FL (US)

(21) Appl. No.: 18/555,981

(22) PCT Filed: Apr. 18, 2022
(86) PCT No.: PCT/US2022/025243

§ 371 Date: Oct. 18, 2023

Related U.S. Application Data
(60) Provisional application No. 63/201,239, filed on Apr. 20, 2021.

(51) Int. Cl. A47K 5/12; A61L 2/10; A61L 2/24

(52) U.S. Cl. CPC A47K 5/1217; A61L 2/10; A61L 2/24; A61L 2202/11; A61L 2202/14

(57) ABSTRACT

A fluid dispenser (e.g., for soap and/or antibacterial solution) is automated and touchless (e.g., operates without contact from the user) to inhibit (e.g., prevents) transfer of bacteria or pathogens via touch. The dispenser delivers an amount of fluid (e.g., soap and/or antibacterial solution) to a user when they place their hands under the dispensing nozzle via a non-drip valve. The dispenser is automated to take temperature readings of users to indicate whether the user has an elevated temperature, which may indicate the user is possibly ill even if not showing any symptoms. The dispenser optionally includes an automated system to sterilize items (e.g., keys, phone, etc.) using ultraviolet (UV) light. The dispenser optionally is automated to perform facial recognition of users and communicate recognition data wirelessly to a computer. The dispenser optionally includes an air odorant system.

PRIORITY AND Incorporation by reference to application(s) This application claims priority to U.S. Provisional Patent Application No. 63/201,239 filed on Apr. 20, 2021 and titled “ANTIBACTERIAL FLUID DISPENSING AND TEMPERATURE SCANNING DEVICE”. The entire content of the above-referenced application is hereby expressly incorporated herein by reference in its entirety for all purposes.

### BACKGROUND

### Field

The present disclosure is directed to a dispenser (e.g., for soap and/or antibacterial solution) and more particularly to an automated fluid dispenser (e.g., for soap and/or antibacterial solution) that may integrate additional capabilities including a temperature scanner, UV sanitizer, and air aromatic system, surveillance node or system, and/or an access control node or system.

### Description of the Related Art

Dispensers for soap and antibacterial solutions are typically manually operated (e.g., have a pump that a user presses upon) to dispense the solution, which can expose a user to bacteria or other pathogens when they contact the pump or other portion of the dispenser to actuate it. Another drawback of existing dispensers is that they often drip solution, leading to spills that need to be regularly cleaned up. Another drawback of existing dispensers is that even when fitted with anti-drip valves or other anti-drip mechanisms, solution will present and accumulate around the dispensing aperture after any number of actuations. This can result in degradation in supplying the solution upon activation. As well, the presence of such solution residue or accumulation may serve as a visual distraction and erode the confidence of the user in the dispenser's standards for sanitization and cleanliness.

Temperature measurements are often an indication of illness (e.g., fever, Covid-19 infection). However, such temperature readings require the use of a separate thermometer

### SUMMARY

In accordance with one aspect of the disclosure, an improved fluid dispenser (e.g., for soap and/or antibacterial solution) is provided that is automated and touchless (e.g., does not require the user to contact or touch any portion of the device), which advantageously inhibits (e.g., prevents) the transfer of bacteria or pathogens via touch. The dispenser can inhibit (e.g., prevent) dripping of fluid (e.g., soap and/or antibacterial solution) after use. The dispenser can vary the volume of fluid supplied per activation based on non-contact, sensed gesture-driven or position-driven actions by the user.

In accordance with another aspect of the disclosure, an improved soap and/or antibacterial dispenser is provided that is automated to take temperature readings of users to indicate whether the user has an elevated temperature, which may indicate the user is possibly ill even if not showing any symptoms.

In accordance with another aspect of the disclosure, an improved soap and/or antibacterial dispenser is provided that is automated to sterilize items (e.g., keys, phone, etc.) using ultraviolet (UV) light.

In accordance with another aspect of the disclosure, an improved soap and/or antibacterial dispenser is provided that is automated to perform facial recognition of users and communicate recognition data wirelessly to a computer.

In accordance with another aspect of the disclosure, an improved soap and/or antibacterial dispenser is provided that is automated to perform pattern recognition (e.g., single-dimensional patterns such as a bar code, or two-dimensional patterns such as a QR code) of users and communicate recognition data wirelessly to a computer.

In accordance with another aspect of the disclosure, an soap and/or improved antibacterial dispenser is provided that is automated to perform non-contact (e.g., induction or RFID technology) recognition of users and communicate recognition data wirelessly to a computer.

In one implementation, a fluid dispensing and temperature scanning device (e.g., for soap and/or antibacterial solution) is disclosed. The fluid dispensing and temperature scanning device may include: a canister extending along a first axis; a stem coupled to the canister and extending along a second axis generally parallel to the first axis; an arm coupled to the stem and extending along a third axis transverse to the second axis; and a head coupled to and suspended from the arm, the head comprising: an electronic display one or more temperature sensors, and a dispensing nozzle, wherein the head is configured to dispense an amount of antibacterial fluid via the dispensing nozzle when a user places their hand under and spaced apart from the head.

In another implementation, a system is disclosed. The system may include: a plurality of fluid dispensing and temperature scanning devices (e.g., for soap and/or antibacterial solution); and a computer system, wherein the plurality of antibacterial fluid dispensing and temperature scanning devices are distributed within an area, each antibacterial fluid dispensing and temperature scanning device communicating with the computer system to provide information from one or more sensors.

### BRIEF DESCRIPTION OF THE DRAWINGS

FIG. 1 is a schematic perspective view of a dispensing device.

FIG. 2 is a partial perspective view of an upper portion of the dispensing device of FIG. 1.

FIG. 3 is a side view of the upper portion of the dispensing device of FIG. 1.

FIG. 4A is a schematic top view of a lid of a canister of the dispensing device of FIG. 1.

FIG. 4B is a schematic bottom view of a head of the dispensing device of FIG. 1.

FIG. 5 is a partial schematic side view of a canister of the dispensing device of FIG. 1.

FIG. 6 is a schematic view of an antibacterial fluid delivery system of the dispensing device of FIG. 1.

FIG. 7 is a schematic block diagram of an electronic control system for the dispensing device of FIG. 1.

FIG. 8 is a flowchart of a temperature scanning process performed with the dispensing device of FIG. 1.

FIG. 9 is a flowchart of a UV sterilization process performed with the dispensing device of FIG. 1.

FIG. 10 is a flowchart of a facial recognition process performed with the dispensing device of FIG. 1.

FIG. 11 is a top view of a dispensing device with a cover removed from the canister.

FIG. 12 is a partial perspective view of an upper portion of the dispensing device of FIG. 11.

FIGS. 13A and 13B are partial perspective views of a head and a display of a dispensing device.

FIG. 14 is a partial perspective view of the back of a head of a dispensing device.

FIG. 15A is a cross-sectional view of the arm, stem, and head of a dispensing device.

FIG. 15B is a cross-sectional view of the arm and head of a dispensing device.

FIG. 15C is a partial perspective view of the arm conduit inlets and outlets.

FIGS. 16A-16B are partial perspective views of a portion of the dispensing device and showing the canister as transparent to illustrate an internal height adjustment system of a dispensing device.

FIG. 16C is a top view of a height adjustment system of a dispensing device.

FIG. 17 is a schematic block diagram of an access control system using one or more dispensing devices.

### DETAILED DESCRIPTION OF THE PREFERRED EMBODIMENT

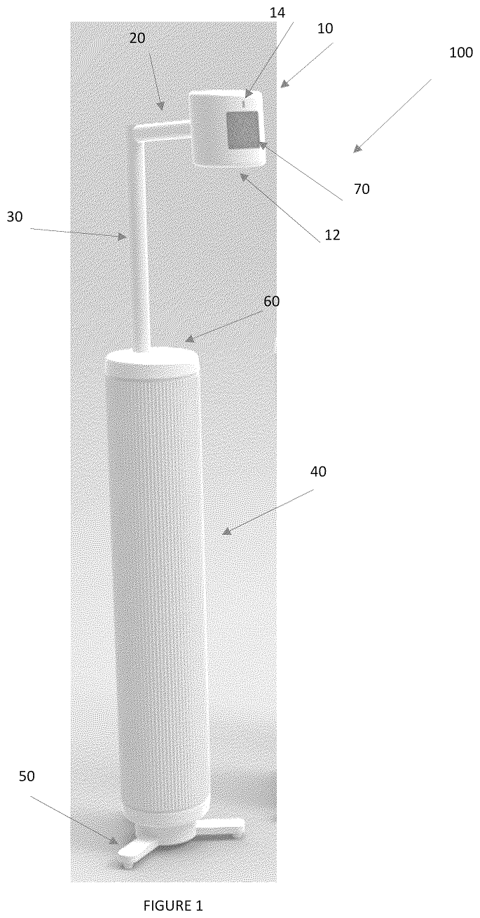

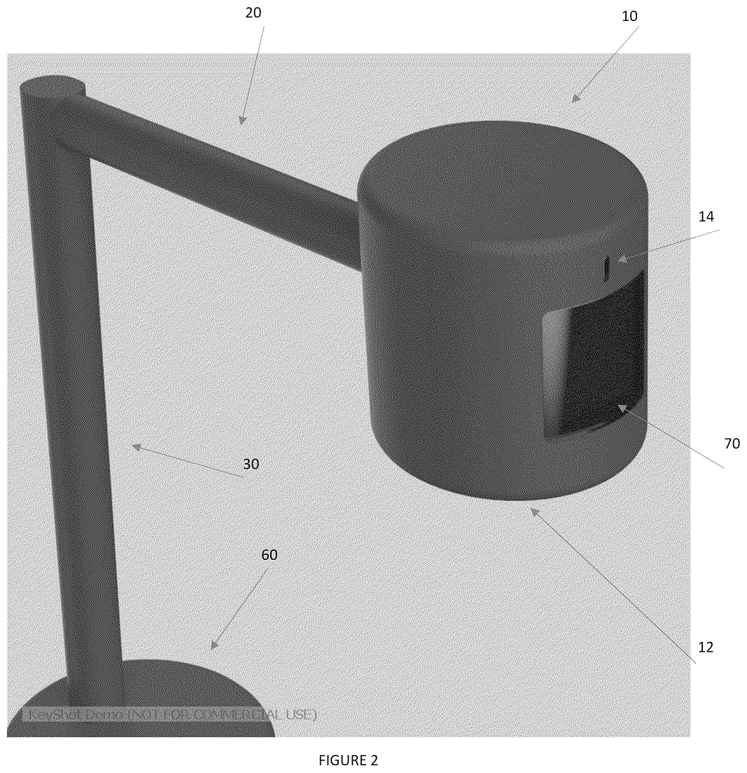

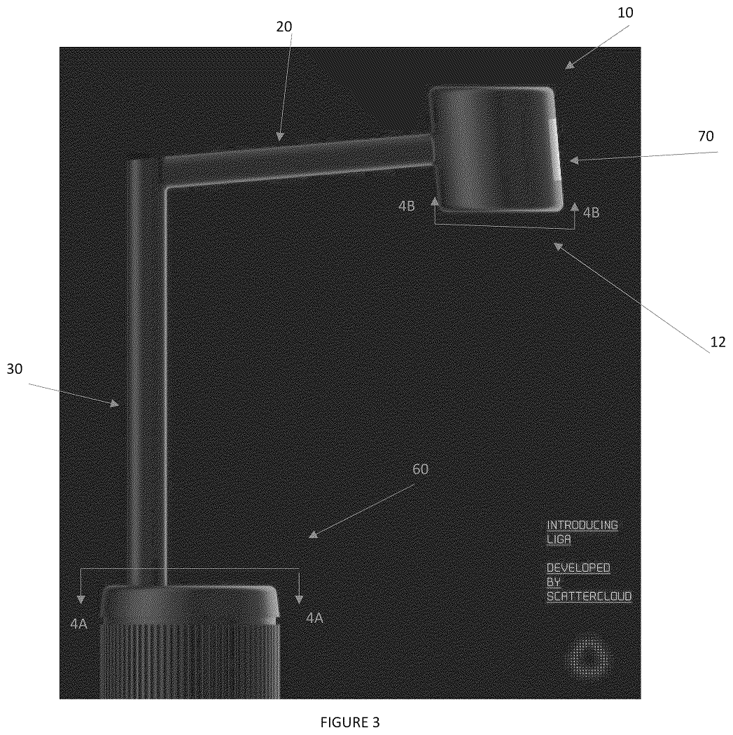

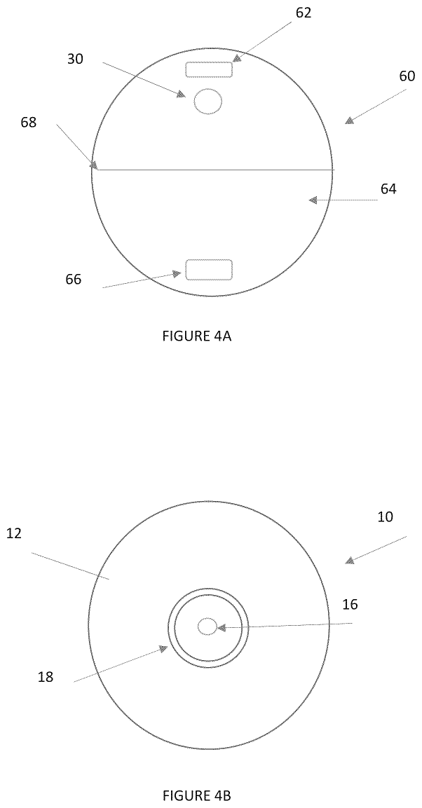

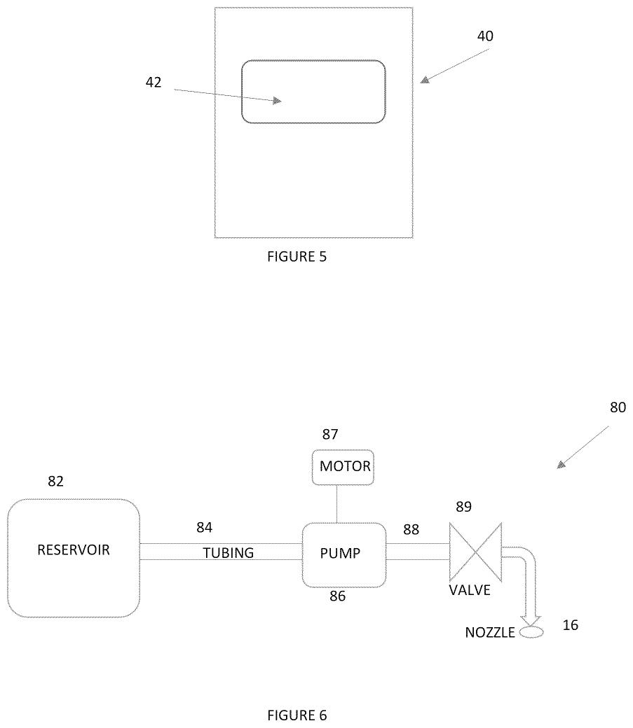

FIGS. 1-3 show a dispensing device 100 that can be operated to dispense a fluid (e.g., a soap and/or an antibacterial fluid, such as a liquid, mist, or foam). In some implementations, the dispensing device 100 can be configured to dispense one or more other solutions. The dispensing device 100 has a head 10 that is supported by (e.g., cantilevered relative to) an arm 20 and stem 30. The stem 30 extends into a canister 40 (e.g., elongate canister, such as cylindrical in shape, that extends along an axis). The canister 40 can be supported (e.g., on a ground surface) by one or more feet or a stand 50. A cover 60 covers an upper end of the canister 40. The head 10 includes a display screen 70 and a port 14 via which temperature sensor readings are taken, as further discussed below. The display 70 can be a general purpose electronic display (e.g., LCD, LED, OLED display, etc.). The head 10 also includes a dispensing nozzle 16 (see FIG. 4B) on an underside 12 of the head 10 for dispensing antibacterial fluid. The head 10 also includes one or more lights 18 (see FIG. 4B) on the underside 12 of the head 10 for illuminating an area under the head 10. In one implementation (see FIG. 4B) the one or more lights 18 are arranged about the dispensing nozzle 16. In one implementation, the one or more lights 18 are light emitting diodes (LEDs). While FIG. 4B illustrates the lights 18 near the center of the underside 12, it is recognized that the lights 18 may be positioned anywhere on the underside 12. For example, the lights 18 may be positioned around the circumference of the underside 12 (e.g., a lower circumferential edge of the head 10) in order to provide greater light dispersion on the floor. In some implementations, the lights 18 may serve one or more purposes. For example, the lights 18 may be used to inform a user or operator that the dispensing device 100 is on and/or operational (e.g., by being turned on, illuminating at a particular color, such as green, etc.). While a user may not be able to identity if the dispensing device 100 is operational based on the display 70 from a distance, the lights 18 may provide an improved indication that the dispensing device 100 is operational. In another example, the lights 18 may provide a visual indication that the dispensing device 100 is about to dispense solution, such as, for example, by pulsing prior to dispensing solution. In another example, the light 18 may be used to cast a glow or light on the floor. Lighting the floor may allow the dispensing device 100 to act as path lighting and/or low-level ambient light source, which may be beneficial if, for example, the dispensing device 100 is positioned outdoors or in an area of low light.

The stem 30 is movably coupled to the canister 40 to adjust a length of the stem 30 above the cover 60 and thereby adjust the height of the head 10 relative to a support surface (e.g., relative to the stand 50). An adjustable height may provide an advantage of allowing the dispensing device 100 to be at a customizable height for multiple users. In one implementation, the stem 30 moves relative to the canister 40 via a rack and pinion system (not shown), where one or more (e.g., two) pinions engage the rack, which is attached to or part of the stem 30 to lock a position of the stem 30 (e.g., relative to the canister 40). In another implementation, the mechanism that allows the stem 30 to move relative to the canister 40 may be a linear rack and gate to allow adjustment/positioning of the head 10 relative to the canister 40. The position of the head 10 relative to the canister 40 can be adjusted manually by unlocking the stem 30 and moving the stem 30, arm 20 and/or head 10 relative to the canister 40. In one implementation, a button 62 (See FIG. 4A) on the cover 60 of the canister 40 (e.g., located behind the stem 30) can be actuated (e.g., pressed, contacted) by a user to unlock the stem 30 (e.g., to disengage the one or more pinions from the rack). In one implementation, the button 62 is a Deadman actuator that the user must continue to press and/or contact to allow adjustment of the stem 30, where upon release of the button 62 the stem 30 automatically locks (e.g., the one or more pinions engage the rack) to lock a position of the head 10 relative to the canister 40.

In one implementation, the dispensing device 100 can have an overall height adjustable between about 36 inches and about 54 inches. In one implementation, the canister 40 can have a length (e.g., height from a bottom of the canister to a top of the canister) of approximately 33 inches, and a height above the ground surface (e.g., height including the stand 50) of about 36 inches.

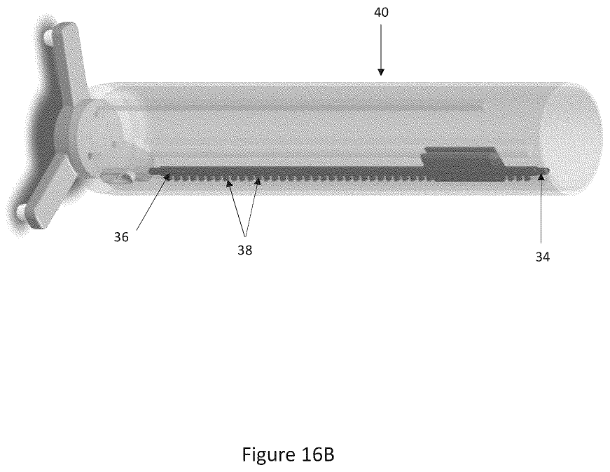

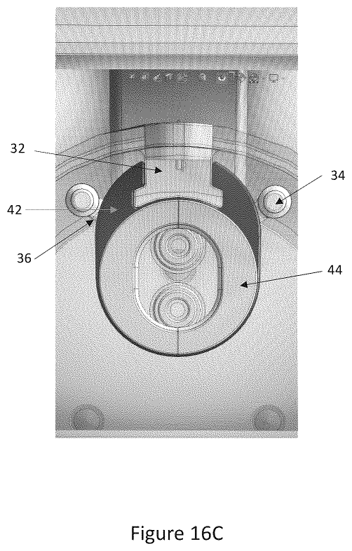

In one implementation, the height of the dispensing device 100 can be adjusted by moving the stem 30 up and down using a height adjustment system that includes a sled on a guide rail as shown in FIGS. 16A-16C. As shown, the height adjustment system may include a guide rail 32 on the inside of the cannister 40 that the stem 30 is moveably connected to by carriage 42. The height adjustment system may also include one or more rotatable bars 34 that each include ladders 36 that contain one or more teeth 38. For example, each ladder 36 may include 2 teeth, 4 teeth, 10 teeth, 15 teeth, 30 teeth, and/or the like. The carriage 42 includes one or more recesses (not shown) that correspond to and are configured to accept the teeth 38 (e.g., to provide a key-slot locking mechanism). The rotatable bars 34 may be spring loaded (e.g., urged toward a default position where the teeth 38 engage the recesses) such that when a user presses a button on, for example, cover 60 (or elsewhere on the device 100, such as the canister 40), the rotatable bars 34 rotate in a direction away from the guide rail 32 (e.g., to disengage the teeth 38 from the recesses). In operation, when a user wants to adjust the height of the dispensing device 100, the user may press a button on the device that causes the spring-loaded rotatable bars 34 to rotate outwards, disengaging the teeth 38 from their corresponding recesses on the carriage 42. Once the teeth 38 are disengaged from the carriage 42, the user can slide (e.g., manually adjust) the stem 30 up or down the guide rail 32 to a desired height. When the user has adjusted the stem 30 to a desired height, the user may disengage the button, which causes the rotatable bars 34 to rotate inward towards the rail wherein the teeth 38 may reengage the corresponding recesses on the carriage 42.

In some implementations, the height adjustment system may also include a rotation component 44 that can include a rotational bearing that allows rotation of the stem 30 about its axis in either direction (e.g., manual rotation of the stem 30 by the user). For example, the stem 30 may be able to rotate 30, 45, 90, 150, and/or the like degrees both clockwise and counterclockwise. Rotation of the stem 30 also causes the arm 20 and head 10 to rotate to the same angle, which may provide an advantage of allowing the dispensing device 100 to be more easily stored. The rotation component 44 may be coupled to the stem 30 and the carriage 42 may be configured to receive the rotation component 44 in order to connect to the stem 30. The dispensing device 100 can be used in a commercial facility (e.g., an office building, hotel), where multiple devices can be placed around the facility (e.g., at entrance(s), at exits, in a lobby, near elevators, etc.) where people can utilize the device 100. The dispensing device 100 can also be used in a residential facility (e.g., single family home, apartment building).

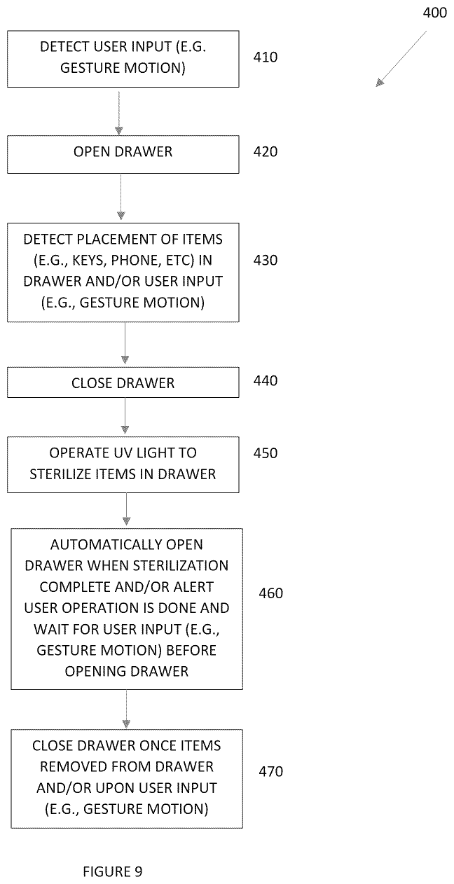

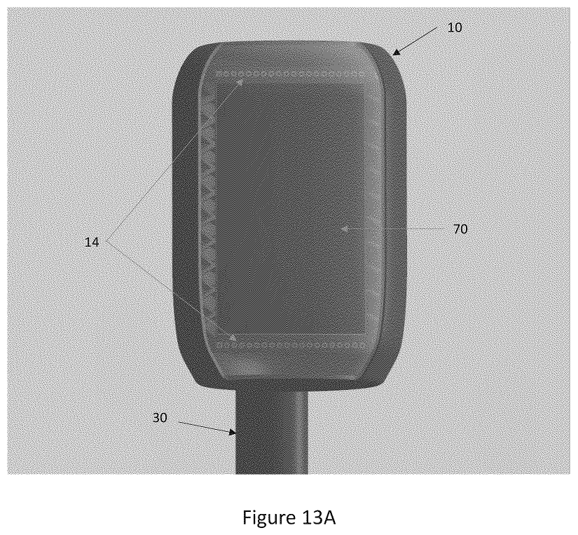

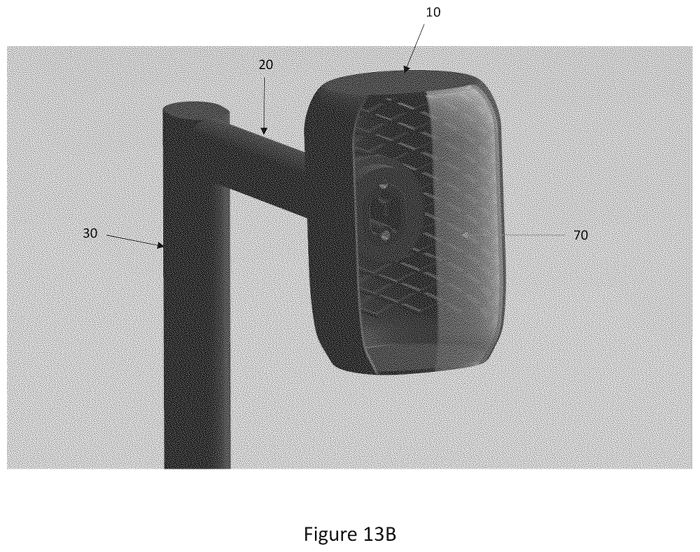

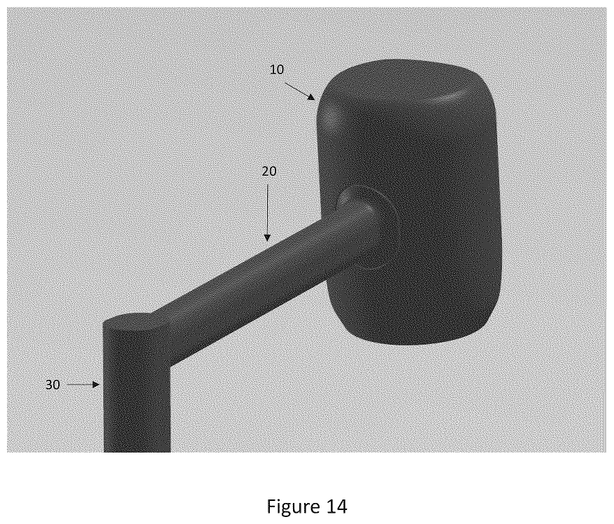

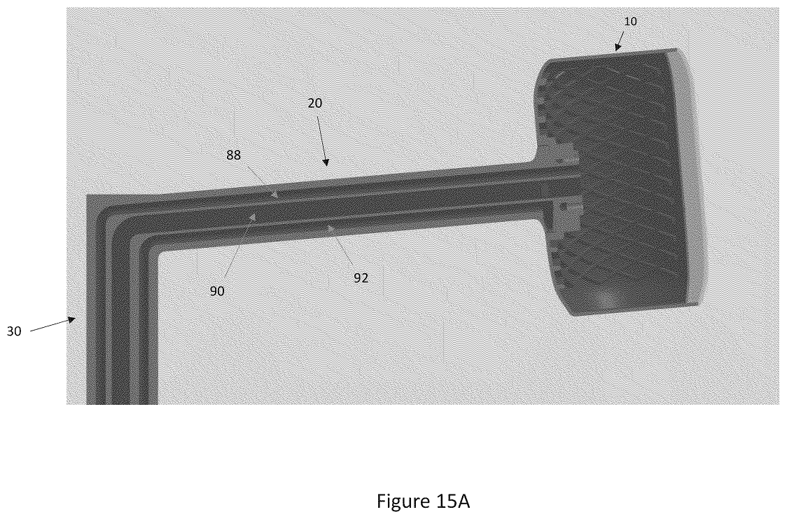

FIG. 11 shows a top view of an embodiment of the dispensing device 100 illustrating an embodiment of the head 10. FIG. 12 shows a partial perspective view of an embodiment of the dispensing device 100 illustrating an embodiment of the head 10. FIG. 14 shows a partial perspective view of the back of the head 10 of an embodiment of the dispensing device 100. In some embodiments, the head 10 may include a display 70 that covers almost the entire front face of the head 10 as shown more clearly in FIGS. 13A and 13B. A large display 70 may provide an advantage of allowing users of the dispensing device 100 to more easily view the information displayed on the display 70. An additional advantage may be that a larger display 70 may allow for more sensors to be contained and utilized in the head 10. For example, as described further herein, the head 10 may include one or more of: one or more temperature sensors, one or more proximity sensors, one or more infrared sensors, one or more time of flight sensors and/or the like. The one or more proximity sensor may include, for example, a laser-based range finder. The sensors are described in more detail with reference to FIG. 7.

In some embodiments, the display 70 may comprise a touch screen. For example, a user of the dispensing device 100 may be able to interact with the device by touching the display 70. Interactions with the dispensing device 100 may include, for example, checking the battery life, checking the reservoir volume, adjusting the settings of the dispensing device 100, and/or the like. In some embodiments, the head 10 may include one or more cameras. The one or more cameras may be used to generate a display of a person on display 70 for optimized temperature readings, read machine-readable optical labels such as barcodes and QR codes, provide facial recognition for an access control system, and/or the like.

FIG. 4A shows a top view of the cover 60 of the canister 40. In one example, the cover 60 has a lid 64 that movably connects to the rest of the cover 60, for example via a hinge 68. In one implementation, a button 66 (See FIG. 4A) on the cover 60 of the canister 40 can be actuated (e.g., pressed, contacted) by a user to unlock the lid 64 relative to the rest of the canister 40. Underneath a the lid 64 (once opened), a locked cap is exposed (e.g., to prevent tampering with the reservoir or antibacterial fluid therein), which can be opened with a key to remove a reservoir 82 that houses the antibacterial fluid from the canister 40. In one example, the removed reservoir 82 can be taken to another location (e.g., elsewhere in the building or hotel) and once filled reinserted into the canister 40 and the lock cap engaged and the lid 64 closed. In another example, the reservoir 82 may be filled without removal and once filled the lock cap engaged and the lid 64 closed. In another example, the removed reservoir 82 is replaced with a filled reservoir 82 (e.g., filled with antibacterial fluid) and the lock cap engaged and the lid 64 closed.

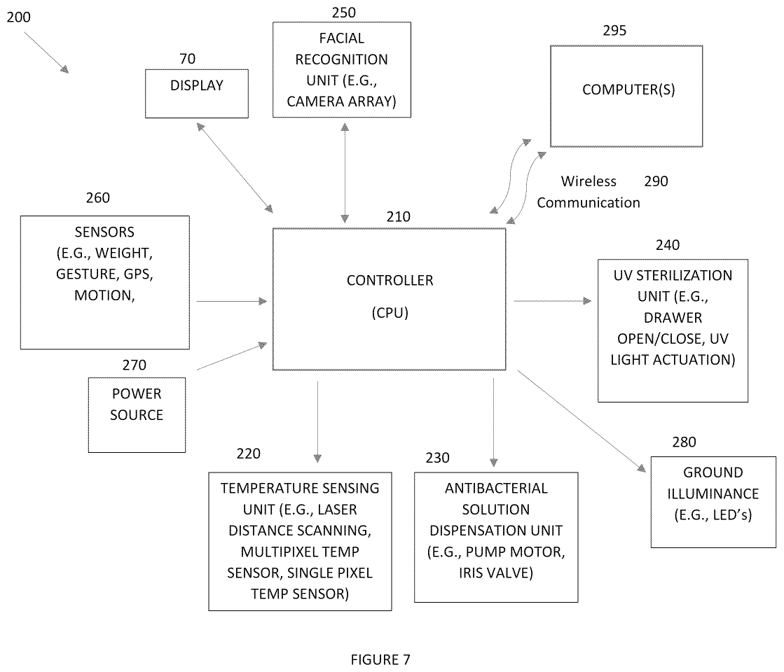

With reference to FIG. 5, in one implementation the dispensing device 100 can have a drawer 42. For example, the drawer 42 can be in the canister 40. The drawer 42 can be part of a UV sterilization unit 240 (See FIG. 7) operated to sterilize items (e.g., keys, phone, etc.) placed in the drawer 42. Once the drawer 42 is closed (e.g. via sensed gesture motions by a user), a UV light (not shown) can be operated (e.g., automatically) to expose the items in the drawer 42 to UV light to sterilize them. The duration of the sanitization process (e.g. actuation of the UV light) can be adjusted (e.g. via sensed gesture motions by a user). The drawer 42 can then be opened (e.g. via sensed gesture motions by a user) for the sterilized items to be withdrawn. The operation of the UV sterilization unit 240 is further described below.

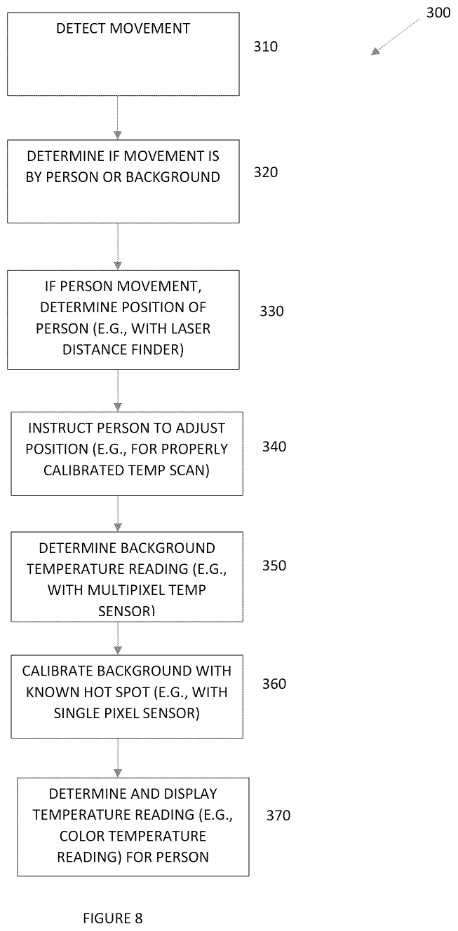

FIG. 6 shows one implementation of a fluid delivery system 80 of the dispensing device 100 (e.g., for delivering antibacterial fluid). The reservoir 82 (once installed in the canister 40) is in fluid communication with a pump 86 via one or more tubes 84. On an underside of the reservoir 82 (e.g., in the canister 40) a sensor (e.g., weight sensor) can sense a weight of the reservoir 82 and communicate information indicative of said weight, which correlates to volume of fluid (e.g., antibacterial fluid) in the reservoir 82 that indicates a fill level or percentage of the reservoir 82. In another implementation, one or more ultrasonic sensors can be used to monitor the fluid level in the reservoir 82. Such information from the sensor(s) can be used to determine if the reservoir 82 needs to be refilled or replaced. The pump 86 can be driven by a motor (e.g. electric motor) 87 and can be self-priming. The pump 86 pumps fluid from the reservoir 82 in the canister 40 via the tubes 84 to the to the head 10. The pump 86 is in fluid communication with a valve 89 (e.g., anti-drip valve) downstream of the pump 86 (e.g., via one or more other tubes 88) that minimizes (e.g., prevents) dripping. In one implementation, the valve 89 can be an iris-type valve, which advantageously inhibits (e.g., prevents) dripping or spillage of the fluid (e.g., antibacterial fluid) from the dispensing nozzle 16 once the valve 89 has been closed. The valve 89 can be electrically actuated. In another implementation, the valve 89 can be an umbrella valve, duckbill valve, and/or the like. In one implementation, the pump 86, motor 87 and valve 89 are located in the head 10, and the one or more tubes 84 extend through the arm 20 and stem 30 and into the canister 40. Optionally, a spillage catch (not shown) can be deployed under the head 10 to capture dripping or spillage of antibacterial fluid dispensed by the fluid delivery system 80.

In some implementations, the dispensing device 100 may include a self-cleaning system. The self-cleaning system may be configured to periodically flush the dispensing system and/or fluid delivery system 80 to prevent residue build-up. The self-cleaning system may include, for example, further piping and valves, as well as a dispensing aperture.

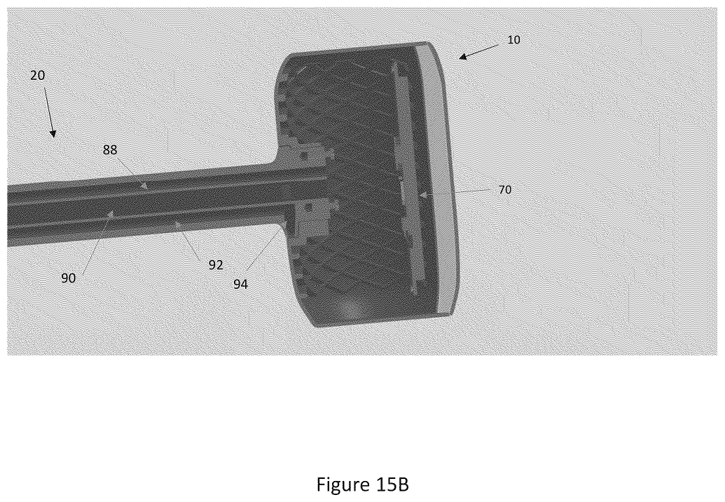

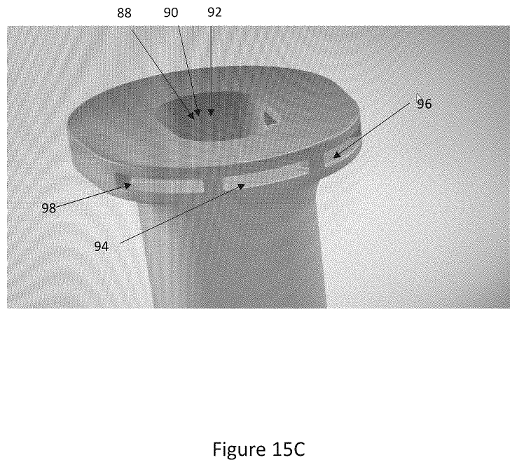

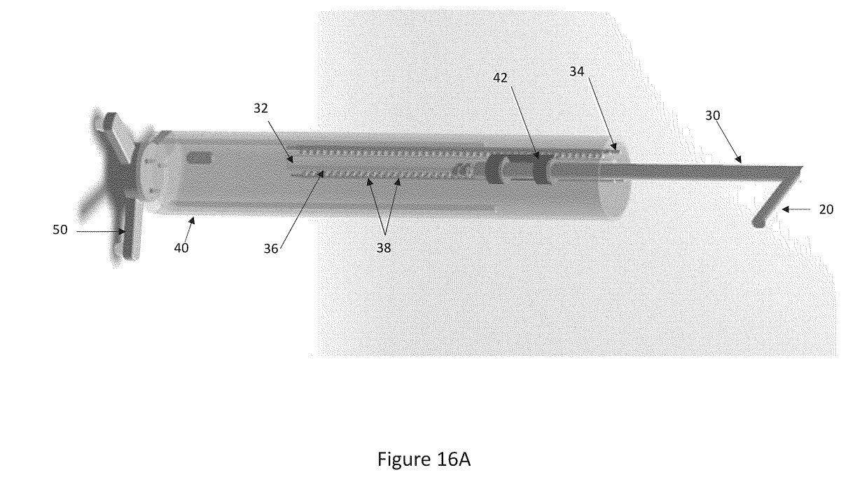

FIGS. 15A and 15B show a cross sectional view of one implementation of a head 10, arm 20, and stem 30 of dispensing device 100. As shown, the arm 20 and the stem 30 include tubes/conduits 88, 90, and 92 that lead to the head 10. In some implementations, the arm 20 and the stem 30 may include more or less conduits than those shown in FIGS. 15A and 15B. As described above, conduit 88 (e.g., upper conduit) may provide a channel for directing the antibacterial solution to the head 10. Conduit 90 (e.g., a center conduit or conduit extending along a central axis of the arm 20) may provide a channel for routing the electrical connections/wires for the dispensing device 100 to the head 10. Conduit 92 (e.g., lower conduit) may provide a channel for an air odorizing system to the head 10 as described below. In some implementations, Conduit 90 may also serve as an air inlet to the reservoir 82 and/or the cannister 40 as described further herein.

In some implementations the dispensing device 100 may include an air odorizing system. The air odorizing system may include a separate reservoir stored in the cannister 40, one or more conduits, such as conduit 92 connected to the separate reservoir, and an outlet (e.g., orifice) 94 in the arm 20 or head 10 that is configured to dispense fluid stored in the separate reservoir. The outlet 94 can be seen in FIG. 15C. In some implementations, the fluid stored in the air odorizing system may be an air freshener fluid, an adsorbent fluid, an oxidizing fluid, one or more fragrances, and/or the like. The air odorizing system may be used to add scents to the air where the dispensing device 100 is located, which may provide an advantage of reducing the need for additional air freshening devices, such as, for example, for hospitality use. In some implementations, the air odorizing system may periodically dispense air odorizing fluid in timed increments, such as, for example, every 1 minute, 2 minutes, 5 minutes, 15 minutes, 30 minutes, 60 minutes, and/or the like. In some implementations, the time of dispersion may be selectable by a user.

FIG. 15C is a partial perspective view of the arm 20 showing conduit inlets and outlets. As shown, the arm 20 may include one or more outlets/inlets 94, 96, and 98. Outlet 94 is an outlet for the air odorizing system as described above. Inlets 96 and 98 may operate as air inlets to the reservoir 82 and/or cannister 40. For example, inlets 96 and 98 may allow the dispensing device 100 to intake environmental air to enter the reservoir 82 to replace the fluid removed from the reservoir 82 when antibacterial solution is dispensed. In some implementations, the inlets 96 and 98 may connect to the conduit 90 such that conduit 90 serves as a multi-purpose passage. For example, conduit 90 may operate as an air inlet to the reservoir 82 while also operating as a routing channel for electrical and communications wiring. In some implementations, the inlets 96 and 98 may be directed at a diagonal downward angle from the arm 20 or the head 10. In some implementations, the inlets 96 and 98 may allow the antibacterial solution dispensation unit 230 to function properly when the reservoir 82 is sealed and locked. In some implementations, the dispensing device 100 may include one or more additional channels that connect the reservoir 82 to inlets/outlets in the arm 20 or the head 10. In some implementations there may only be one air inlet, while in other implementations, there may be more than two air inlets.

FIG. 7 shows one implementation of a control system 200 for the dispensing device 100. The control system 200 includes a controller or CPU 210, which can include one or more processors. The controller 210 can be mounted on a printed circuit board (PCB) along with other electronics and can be located in the head 10. The controller 210 can control an operation of a temperature sensing unit 220 as further described below. The controller 210 can also control the antibacterial solution dispensation unit 230 (e.g. fluid delivery system 80). For example, the controller 210 can control the operation of the motor 87 and/or valve 89 to dispense antibacterial fluid. The controller 210 can also control a UV sterilization unit 240. For example, the controller 210 can control the opening and closing of the drawer 42 and the operation of the UV light(s). The controller 210 can also control a facial recognition unit 250 (e.g., one or more cameras or a camera array) as further described below. The controller 210 can communicate with one or more sensors 260. The one or more sensors 260 can include a weight sensor (e.g., for sensing a weight or volume of the reservoir 82 indicative of a fill level of the reservoir 82), a gesture sensor (e.g., for sensing gesture motions by a user to open and/or close the drawer 42), a global positioning system (GPS) sensor for determining and/or tracking a geolocation of the dispensing device 100, a motion sensor (e.g., for sensing the presence of a person in front of the dispensing device 100 to actuate the temperature sensing unit 220 and/or facial recognition unit 250), and a proximity sensor (e.g., for sensing a user's hands under the head 10 to actuate the antibacterial solution dispensation unit 230). The one or more sensors 260 can also include a tilt sensor, an ambient light sensor, an ambient temperature sensor, a proximity sensor (e.g., underside of head 10, such as to sense motion or presence of user hands to dispense antibacterial fluid), a temperature sensor array (e.g., single pixel sensor), a temperature sensor array (e.g., multi-pixel sensor (motion sensing)), a temperature sensor array (e.g., wide-array sensor), a time of flight sensor (e.g., senses the distance to a user to ensure they are positioned with the valid temperature sensing range, and/or senses the distance of a user's hands), a camera or camera array sensor (e.g., Facial Recognition System, 1D or 2D Pattern Recognition System, and reading machine-readable optical label), an RFID or inductive sensor (e.g., for a Non-Contact Recognition System), a gesture sensor, a load/strain sensor (e.g., for sensing weight of reservoir 82), and a GPS sensor. Other sensors are possible. The controller 210 also communicates with the display 70 in the head 10 (e.g., to communicate a battery level, a reservoir fill level, and/or a temperature reading) for display of information on the display 70. The controller 210 (and other electronics) of the dispensing device 100 receive power from a power source 270. The power source 270 can be wall power that is provided to the dispensing device 100 via a cable that connects to a connector (e.g., USB-c connector) of the device 100 (e.g., on the canister 40). For example, the connector (e.g., USB-c connector) of the dispensing device 100 can receive 5 V converted from line voltage. Additionally or alternatively, the dispensing device 100 can have one or more batteries that provide power to the controller 210 and other electronics (e.g., when the cable is not connected between the dispensing device 100 and wall power). In one implementation, when the cable connects the dispensing device 100 to wall power, the power delivered to the dispensing device 100 is used to charge the one or more batteries, and the one or more batteries power the controller 210 and electronics. The one or more batteries can be rechargeable and replaceable; for example, the one or more batteries can be housed in the canister 40 (e.g., accessible via the lid 64). In another implementation, wall power directly powers the controller 210 and electronics when the cable connects the dispensing device 100 to wall power.

In some embodiments, the head 10 may include a proximity sensor and/or a time of flight sensor to facilitate the dispensation of the fluid (e.g., soap and/or antibacterial solution). For example, these sensors may be positioned on the underside 12, near the bottom of display 70, and/or the like. As described herein, the control system 200 may control the dispensation unit 230 and communicate with the sensors to determine whether an object, such as a user's hand(s), are positioned under the head 10 and the distance to the object. Based on this information, the control system 200 may provide a variable dispensation of the fluid (e.g., soap and/or antibacterial solution). For example, if the user's hand(s) are closer to the underside 12, as determined by the time of flight sensor (e.g., proximity sensor), the control system 200 may instruct the dispensation unit 230 to dispense a small amount of the fluid (e.g., soap and/or antibacterial solution). Conversely, if the user's hand(s) are further away from the underside 12, the control system 200 may instruct the dispensation unit 230 to dispense a larger amount of the fluid (e.g., soap and/or antibacterial solution). In some embodiments, the amount of fluid (e.g., soap and/or antibacterial solution) dispensed may directly correspond to the distance of the user's hands. In other embodiments, the amount of fluid (e.g., soap and/or antibacterial solution) dispensed may correspond to ranges of hand distances. For example, a small amount of solution for hand distances between zero and six inches, a larger amount of solution for hand distances between six and twelve inches, an even larger amount of solution for hand distances between twelve and eighteen inches, and so forth. In some embodiments, the distance scale may be reversed. For example, more solution may be dispensed for closer hand distances while less solution is dispensed for further hand distances. In some implementations, the variable dispensation may be controlled by controlling the motor 87. In other implementations, the variable dispensation may be controlled by controlling the valve 89.

With continued reference to FIG. 7, the controller 210 can communicate wirelessly 290 with one or more computers 295 (e.g., remote computers), such as via Wi-Fi (e.g., the controller 210 can include a transceiver). For example, in a commercial setting (e.g., hotel, building), the controller 210 can communicate information (e.g., fill level of reservoir 82, temperature readings of users, facial recognition information) to one or more computers in a control room, where decisions can be made based on the communicated data (e.g., allow or disallow entry to an individual based on a temperature reading indicating that the individual is ill or if facial recognition indicates that the individual is not authorized to have entry). Such communicated data can also give an indication of usage rates of the dispensing device 100, allow evaluation of trends at the location of the dispensing device 100 (e.g., number of people with elevated temperatures), and monitor health of the dispensing device 100 (e.g., if reservoir 82 needs refilling). Anonymized data can also be communicated by the dispensing device 100 to a computer 295 (e.g., at the service provider) to track hot spots (e.g., of individuals with elevated temperatures that may indicate increase in potential illness) and provide an early warning system for potential illness.

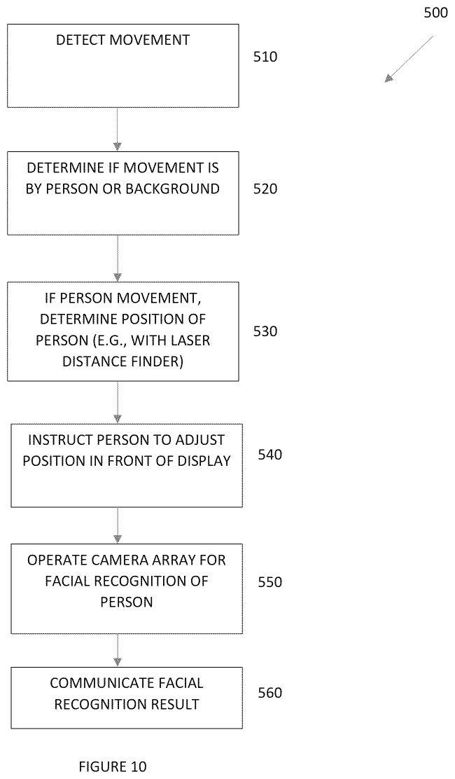

FIG. 8 shows a method or process 300 for performing temperature scans with the dispensing device 100 (e.g., with the temperature sensing unit 220). In one implementation, in a normal operating mode, the display 70 displays an indication of power level/battery life, an indication of an amount of antibacterial fluid remaining in the reservoir 82 (e.g., a percentage of fill level of the reservoir 82), a current time, and/or the like. The method 300 includes the step of detecting a movement 310 in front of the dispensing device 100 (e.g., in front of the display 70), followed by a determination 320 if the detected movement is of a person or background movement. If the detected movement is of a person within a certain threshold, the method 300 also includes the step of determining (e.g., with a laser distance finder that directs a laser via the port 14) a position of the person (e.g., position in front of the display 70), followed by the step of instructing or alerting 340 the person to adjust their position in front of the dispensing device 100 (e.g., in front of the display 70) to provide a properly calibrated temperature reading of the person. For example, once it is determined that a person is within a certain threshold, the display 70 may change from a normal operating mode to an instruction mode. An instruction mode may include visual indications on display 70 to instruct/provide feedback to a person to change their position and/or angle with respect to the head 10 to optimize the temperature reading. An optimized temperature reading may require that a user be within a certain horizontal angle range, vertical angle range, and/or distance range with respect to the head 10. For example, an optimized horizontal angle range may be 160 degrees+/−five degrees, an optimized vertical angle range may be 160 degrees+/−five degrees, and an optimized distance range may be between, for example, six and thirty-six inches. However, it is recognized that the ranges for an optimized temperature reading may vary based on the optic system and sensors used in the dispensing device 100. Visual indications on the display 70 may include using one or more cameras to generate and display an image of the person as well as a location of where the temperature reading is currently directed. Based on this display, a person can adjust their position in front of the display 70 so that the position of the temperature reading is in an optimized location, such as, for example, their forehead, their wrist, and/or the like. Providing a visual display of the person's position may provide a benefit of allowing the dispensing device 100 to quickly conduct a temperature reading because a person has immediate feedback of how their movement affects the temperature reading. The method 300 also includes the step of determining 350 a background temperature reading (e.g., with a multi-pixel temperature sensor in the head 10 that takes the reading via the port 14) that, for example, is used as a baseline reading, followed by the step of calibrating 360 the background with a known hot stop in the background (e.g., using a single pixel temperature sensor in the head 10 that takes the reading via the port 14). The method 300 also includes determining and displaying 370 (e.g., on the display 70) the temperature reading for the person. The temperature reading can be provided as a color (e.g., color temperature gradient), where green or blue colors are clear areas and orange or red colors are warmer areas (e.g., above a threshold). Advantageously, feedback between the multipixel temperature sensor and single pixel temperature sensor results in a temperature scan for the person that is accurate within ±0.2 degrees Celsius.

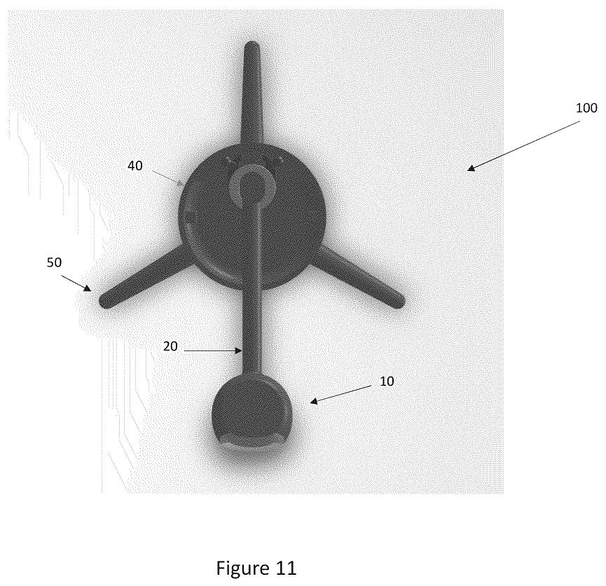

FIG. 9 shows a method or process 400 for performing UV sterilization with the dispensing device 100 (e.g., with the UV sterilization unit 240). The method includes the step of detecting 410 a user input (e.g., a gesture motion from the user, such as from their hand) with one of the sensors 260, followed by the step of opening 420 the drawer 42 based on the user input (e.g., when the correct gesture motion is provided). The method 400 also optionally includes detecting 430 the placement of items (e.g., keys, phone, etc.) in the drawer 42 (e.g., via a weight or load sensor in or adjacent to the drawer 42) and/or based on user input (e.g., gesture motion). The method also includes the step of closing 440 the drawer 42 (e.g., based on a corresponding gesture motion via one of the sensors 260), followed by the step of operating 450 (e.g., automatically) one or more UV lights to sterilize the items in the drawer 42. The method 400 also includes the step of opening 460 (e.g., automatically) the drawer 42 when the sterilization process is complete and/or alert the user (e.g., via the display 70, via an audio signal, wirelessly via the user's smartphone) that the sterilization process has been completed and wait for user input (e.g., a gesture motion) before opening the drawer 42. The method 400 also includes closing 470 the drawer 42 (e.g., automatically) once the items are removed from the drawer 42 (e.g., via signal from weight or load sensor in or adjacent the drawer 42) and/or upon user input (e.g., a corresponding gesture motion).

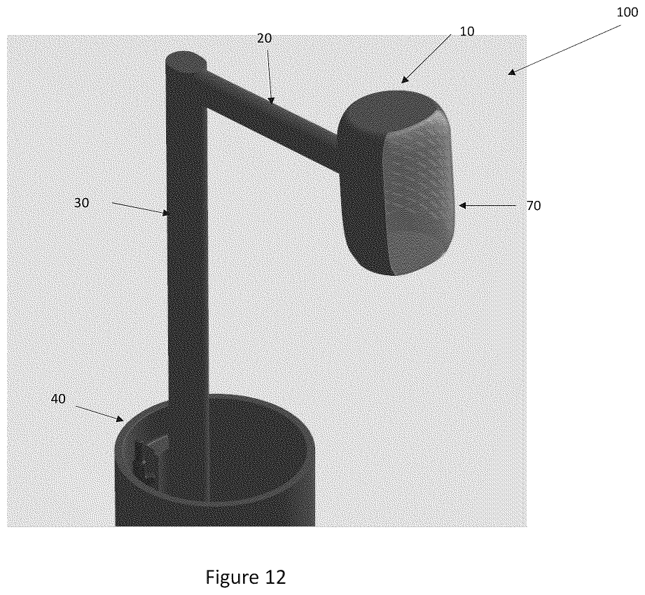

FIG. 10 shows a method or process 500 for performing facial recognition scans with the dispensing device 100 (e.g., with the facial recognition unit 250). The method 500 includes the step of detecting a movement 510 in front of the dispensing device 100 (e.g., in front of the display 70), followed by a determination 520 if the detected movement is of a person or background movement. If the detected movement is of a person, the method 500 also includes the step of determining (e.g., with a laser distance finder that directs a laser via the port 14) a position of the person (e.g., position in front of the display 70), followed by the step of instructing or alerting 540 the person to adjust their position in front of the dispensing device 100 (e.g., in front of the display 70) to provide a proper facial recognition scan of the person. For example, similar to the temperature reading described above, instructing a person to adjust their position may include using the cameras to generate an image of the person for display on display 70 to provide feedback of the current position of the person. The method 500 also includes the step of operating 550 a camera array (e.g., in the head 10 that takes the scan via the port 14) to scan the face of the user in front of the display 70, followed by the step of communicating 560 (e.g., wirelessly to the computer(s) 295) the result of the facial recognition scan. Based on the result of the facial recognition scan, the person can, for example, be granted (e.g., automatically) access to an area (e.g., entrance to a building, to a lobby, to an elevator, etc.) or rejected access if it's determined the person is unauthorized to be in or on the premises.

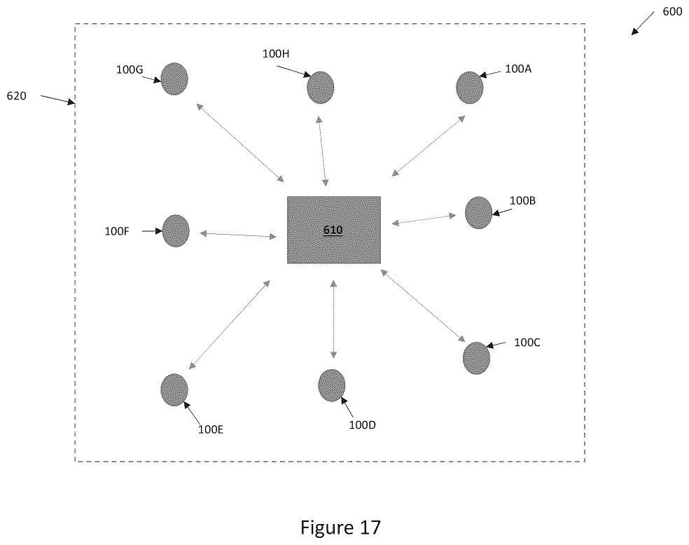

As described with reference to FIG. 10, the dispensing device 100 can be used as part of an access control system by using the facial recognition scans to allow or reject access to certain areas. For example, the dispensing device 100 may communicate with known building control systems to utilize the facial recognition scanning capabilities or work alone as a control system. FIG. 17 illustrates an implementation of an access control system 600 using one or more dispensing devices 100. The access control system 600 may include one or more dispensing devices, for example, dispensing devices 100A-100H (e.g., similar or identical to dispensing device 100) and a computer system 610, operating to control a specific area 620, such as, for example, a hotel lobby, office building lobby, concert hall, and/or the like. In the control system 600, the dispensing devices 100A-100H operate as nodes in the system, with each dispensing device communicating with the computer system 610. For example, if the computer system 610 is at the same location as the dispensing devices 100A-100H, the dispensing devices may communicate over a local network that includes communication with local building systems. In another example, if the computer system 610 is off premise, such as at a server location, the dispensing devices 100A-100H may communicate over a cloud network with the computer system 610. The dispensing devices 100A-100H may provide real-time feedback to the computer system 610 to provide security, access control, and/or the like. For example, the system 600 may allow for live-time surveillance using the dispensing devices 100A-100H or the system 600 may allow for live time access control.

### Additional Embodiments

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z.” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately.” “about.” “generally.” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Of course, the foregoing description is that of certain features, aspects and advantages of the present invention, to which various changes and modifications can be made without departing from the spirit and scope of the present invention. Moreover, the devices described herein need not feature all of the objects, advantages, features and aspects discussed above. Thus, for example, those of skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed devices.

1. A fluid dispensing and temperature scanning device, comprising:
a canister extending along a first axis;
a stem coupled to the canister and extending along a second axis generally parallel to the first axis;
an arm coupled to the stem and extending along a third axis transverse to the second axis; and
a head coupled to and suspended from the arm, the head comprising:
an electronic display,
one or more temperature sensors, and
a dispensing nozzle,
wherein the head is configured to dispense an amount of a fluid via the dispensing nozzle when a user places their hand under and spaced apart from the head.

2. The fluid dispensing and temperature scanning device of claim 1, wherein the one or more temperature sensors include a multi-pixel temperature sensor and single-pixel temperature sensor, the one or more temperature sensors configured to sense a temperature of the user in front of the electronic display to determine if the sensed temperature is above a threshold and to wirelessly communicate the sensed temperature to a remote computer.

3. The fluid dispensing and temperature scanning device of claim 1, wherein the stem is configured to move relative to the canister to adjust a height of the head above a ground surface.

4. The fluid dispensing and temperature scanning device of claim 1, further comprising a UV sterilization unit comprising a drawer selectively openable to receive one or more items and one or more UV lights operable to sterilize the one or more items when the drawer is closed.

5. The fluid dispensing and temperature scanning device of claim 1, further comprising a camera array in the head configured to perform a facial scan of the user in front of the electronic display and to wirelessly communicate information associated with the facial scan to a remote computer.

6. The fluid dispensing and temperature scanning device of claim 1, wherein the fluid is delivered to the dispensing nozzle via an iris valve.

7. The fluid dispensing and temperature scanning device of claim 1, wherein the head further comprises a light on an underside of the head.

8. The fluid dispensing and temperature scanning device of claim 7, wherein the light is configured to pulse prior to the head dispensing the fluid.

9. The fluid dispensing and temperature scanning device of claim 1, wherein the stem is configured to move rotate relative to the canister.

10. (canceled)

11. The fluid dispensing and temperature scanning device of claim 1, wherein the stem and the arm comprise one or more conduits that lead to the head.

12. The fluid dispensing and temperature scanning device of claim 11, wherein a first reservoir is stored in the canister, wherein the first reservoir is configured to store the fluid.

13. The fluid dispensing and temperature scanning device of claim 12, wherein a first conduit of the one or more conduits is configured to transport the fluid from the first reservoir to the dispensing nozzle.

14. The fluid dispensing and temperature scanning device of claim 12, wherein at least one conduit of the one or more conduits is configured to transport environmental air from an orifice to the first reservoir.

15. The fluid dispensing and temperature scanning device of claim 12, wherein a second reservoir is stored in the canister, wherein the second reservoir is configured to store odorizing fluid.

16. The fluid dispensing and temperature scanning device of claim 15, wherein a second conduit of the one or more conduits is configured to transport the odorizing fluid from the second reservoir to an orifice in the arm or the head.

17. (canceled)

18. The fluid dispensing and temperature scanning device of claim 1, further comprising a sensor configured to determine a distance to an object under and space apart from the head, wherein the head is configured to dispense a variable amount of the fluid via the dispensing nozzle based on the distance.

19. The fluid dispensing and temperature scanning device of claim 1, wherein the electronic display is configured to display a first display when the device does not detect movement within a threshold distance of the device.

20. (canceled)

21. The fluid dispensing and temperature scanning device of claim 19, wherein the electronic display is configured to display a second display when the device detects movement within the threshold distance of the device.

22. The fluid dispensing and temperature scanning device of claim 21, wherein the second display comprises a visualization of a temperature reading location on the user.

23. The fluid dispensing and temperature scanning device of claim 21, wherein the second display is configured to instruct the user to adjust their position relative to the head for an optimized temperature reading.

24. (canceled)

25. (canceled)

26.-31. (canceled)
